(19) United States
(12) Patent Application Publication
(10) Pub. No.: US 2006/0095019 A1
(43) Pub. Date: May 4, 2006

(54) Urinary catheter

(76) Inventors: W. Cary Dikeman, Leawood, KS (US); Gordon Edgar Atkinson, Anna Maria, FL (US); Mitchell Wade Yadven, Bradenton, FL (US)

Correspondence Address: STEVENS & SHOWALTER LLP, 7019 CORPORATE WAY, DAYTON, OH, 45459-4238, US

(21) Appl. No.: 10/979,286

(22) Filed: Nov. 2, 2004

(51) Int. Cl. A61M 27/00

(52) U.S. Cl. 604/544; 604/328

(57) ABSTRACT

A urinary catheter including an elongated tube formed of a flexible material. A proximal end of the tube includes an opening placing an exterior of the elongated tube in fluid communication with an interior fluid passage of the elongated tube, where the proximal end defines an inlet end of the elongated tube. A distal end of the tube is formed by the flexible material, and a socket is located within the distal end of the flexible material and defines a fluid outlet for the elongated tube. A valve having a fluid passage therethrough is connected to the socket and includes a movable closure member displaceable to control fluid flow from the elongated tube. The valve is removable from the socket to permit a flushing syringe to be connected in engagement with the socket.

### BACKGROUND OF THE INVENTION

1. Field of the Invention

The present invention relates generally to urinary catheters and, more particularly, to a Foley-type urinary catheter provided with a valve connection end for interchangeably receiving a detachable valve and a flushing syringe or a beside urinary collection bag.

2. Description of Prior Art

The Foley-type catheter has been widely used for many years as part of urinary collection systems and comprises a flow conduit for extending through a patient's urethra, an inflatable balloon located on one end of the flow conduit for positioning and retention in a patient's bladder, and a urine collection bag attached to an extracorporal end of the flow conduit. The Foley-catheter with a drainage bag provides a passive urine collection system which may be used for an extended time, and may comprise a bedside urine collection bag or, for a mobile person, may comprise a drainage bag strapped or otherwise retained to the person, and requires periodic drainage of the bag.

Although the provision of a bag in the catheter system permits mobility of the patient while wearing the catheter, such bags are typically attached to the leg of the patient, limiting his mobility and the type of clothing worn by the patient to clothes that sufficiently cover the bag. Further, catheter systems incorporating bags do not provide a patient with the ability to selectively control the urinary discharge function, and thus do not provide the patient with control over urination in a manner which emulates a normal physiological urination process. Prior patents which provide an alternative to inclusion of a drainage bag in a urinary catheter system include U.S. Pat. Nos. 5,445,626, 5,601,537 and 5,707,357.

There remains a need for a catheter system which permits improved mobility of the patient and which is conveniently operated and maintained.

### SUMMARY OF THE INVENTION

In accordance with one aspect of the invention, a urinary catheter is provided comprising an elongated tube formed of a flexible material. A proximal end of the tube is formed by the flexible material and includes an opening placing an exterior of the elongated tube in fluid communication with an interior fluid passage of the elongated tube, where the proximal end defines an inlet end of the elongated tube. A distal end of the tube is formed by the flexible material, and a socket is located within the distal end of the flexible material and defines a fluid outlet for the elongated tube. A valve having a fluid passage therethrough is connected to the socket and includes a closure member displaceable to control fluid flow from the elongated tube.

In accordance with another aspect of the invention, a urinary catheter is provided comprising an elongated tube formed of a flexible material. A proximal end of the tube is formed by the flexible material and includes an opening placing an exterior of the elongated tube in fluid communication with an interior fluid passage of the elongated tube, where the proximal end defines an inlet end of the elongated tube. A secondary passage extends parallel to the fluid passage and an actuated element is in fluid communication with the secondary passage. The tube includes a distal end formed by the flexible material and a socket is located within the distal end of the flexible material and defines a relatively rigid fluid outlet for the elongated tube.

Other features and advantages of the invention will be apparent from the following description, the accompanying drawings and the appended claims.

### BRIEF DESCRIPTION OF THE DRAWINGS

FIG. 1 is a perspective view of a catheter system illustrating the present invention;

FIG. 2 is a cross-sectional side elevation view of the catheter system of FIG. 1;

FIG. 3 is an exploded perspective view of the catheter system of FIG. 1;

FIG. 4 is a perspective view of a socket element for the catheter system;

FIG. 5 is a perspective view of a valve body for the catheter system;

FIG. 6 is a perspective view of an alternative configuration of the socket element;

FIG. 7 is a perspective view of an alternative configuration of the valve body;

FIG. 8 is a cross-sectional side elevation view of the catheter system with a syringe connected at the socket;

FIG. 9 is a partial anatomical side-sectional view of the urinary tract of a male patient showing the catheter system of FIG. 1 implanted therein;

FIG. 10 is a cross-sectional side elevation view of the catheter system showing an alternative embodiment for the socket without an attachment connected at the socket;

FIG. 11 is a cross-sectional side elevation view of the catheter system of FIG. 10 with a valve connected at the socket;

FIG. 12 is a cross-sectional side elevation view of the catheter system of FIG. 10 with a syringe connected at the socket;

FIG. 13 is an elevation view of an alternative embodiment of a valve for the catheter system of the present invention;

FIG. 14 is a perspective view of the valve body for the valve of FIG. 13; and

FIG. 15 is a perspective cross-sectional view of the valve cap for the valve of FIG. 13.

### DETAILED DESCRIPTION OF THE INVENTION

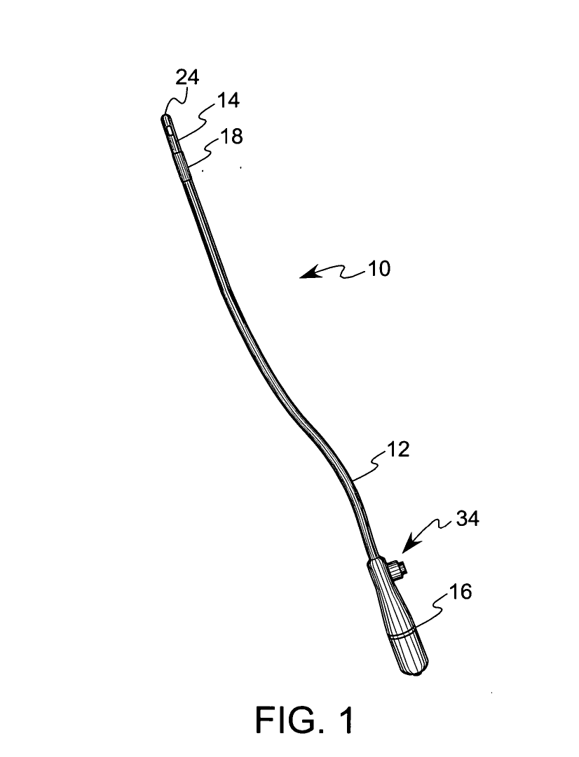

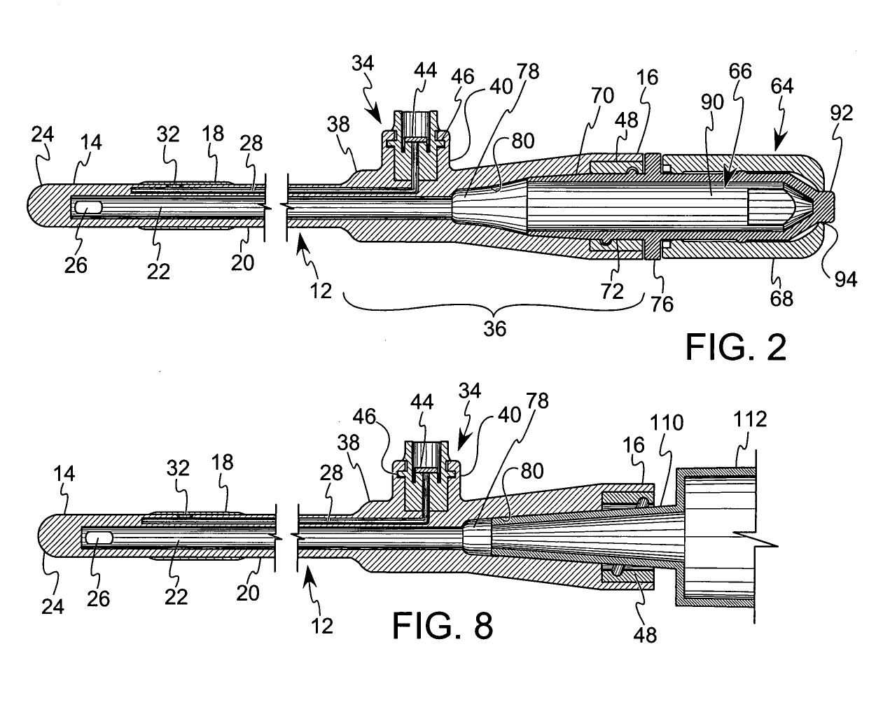

Referring initially to FIG. 1, the urinary catheter 10 illustrating an embodiment of the present invention comprises a catheter body 12 defined by an elongated tube formed of a flexible material and including a proximal end portion 14 and a distal end portion 16. The material forming the catheter body 12 comprises an elastomeric material, such as is known in the art for catheter constructions. An actuated element comprising an expandable balloon 18 is located on the catheter body 12 adjacent the proximal end portion 14 for retaining the catheter within a patient's bladder. This actuated element may be formed as in a conventional Foley catheter with an elastic wall 20 (FIG. 2) defining the expandable balloon 18.

Referring to FIG. 2, the catheter body 12 is formed with a central, first lumen or passage 22 extending from a location adjacent the distal end portion 16 substantially the length of the catheter body 12. The first passage 22 terminates at the proximal end portion 14 in a rounded closed end 24 which facilitates insertion of the catheter 10 into and through the urethra. One or more openings 26, such as Murphy eye openings, are formed on opposing sides of the catheter body at the terminal end of the first passage 22 to provide fluid flow between the bladder and the first passage 22.

The catheter body 12 also includes a secondary lumen or passage 28 which extends within the wall 20 of the catheter body 12 parallel to the first passage 22, such that the catheter body 12 is defined as a double lumen structure. The secondary passage 28 extends from an opening 32 in the wall 20 of the catheter body 12, in communication with the interior area of the balloon 18, to an inflation port 34 located on the catheter body 12 adjacent the distal end portion 16. Fluid may be injected through the inflation port 34 to pass through the secondary passage 28 to inflate the balloon 18 from a fully collapsed configuration to an inflated configuration for retaining the proximal end portion 14 of the catheter body 12 in the bladder.

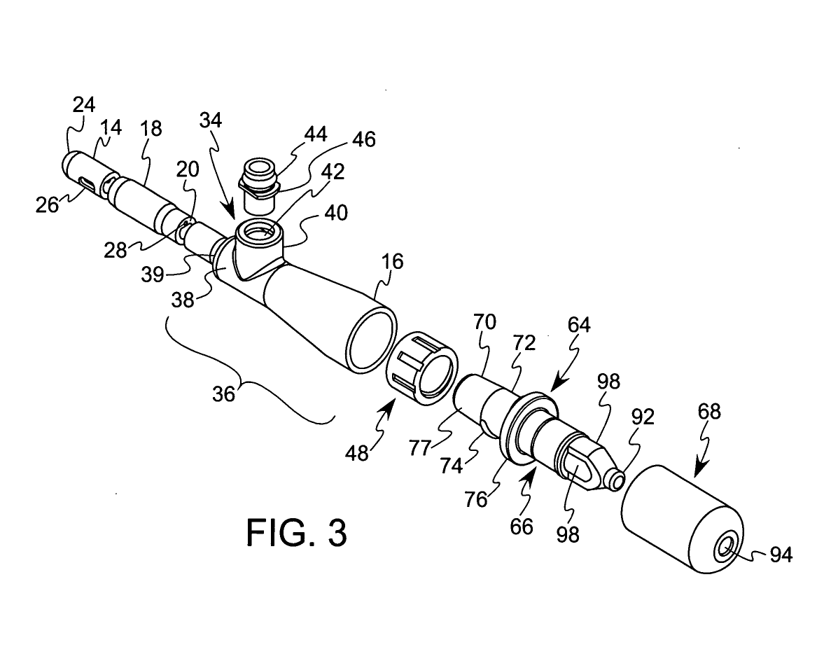

Referring further to FIG. 3, the distal end portion 16 of the catheter body 12 terminates in a hub section 36 defined by an enlarged extracorporal portion 38 of the catheter body wall 20. The inflation port 34 is located at a proximal end 39 of the hub section 36 and comprises an inflation port housing 40 formed integrally with the portion 38 of the catheter wall 20. The inflation port housing 40 includes an aperture 42 for receiving an inflation port valve 44 of conventional design. The aperture 42 is in fluid communication with the secondary passage 28, and the valve 44 comprises a normally closed valve permitting insertion of a syringe for injection of a fluid to inflate the balloon 18. It should be noted that the housing 40 provides a low profile, direct mounting location for the valve 44 to define a compact configuration for the inflation port 34 on the hub section 36 of the catheter body 12. The valve 44 is preferably molded in place during formation of the catheter body 12 and housing 40, and includes a radially extending flange 46 to facilitate retention of the valve 44 within the housing 40.

Referring to FIGS. 2-4, the hub section 36 further includes a socket 48 formed of a relatively rigid material in comparison to the material of the catheter body 12. In one embodiment, the socket 48 is formed of a non-elastic material and may be formed of thermoplastic. The socket comprises a cylindrical body 50 extending around longitudinal axis 52 and including an exterior wall 54, an interior wall 56, and a proximal end surface 58 and distal end surface 60. The interior wall 56 defines a substantially smooth surface extending in a direction from the distal end surface 60 toward the proximal end surface 58, and including a locking structure illustrated as a spiral or threaded groove member 62 formed into the surface of the interior wall 56 extending radially outwardly from the longitudinal axis 52.

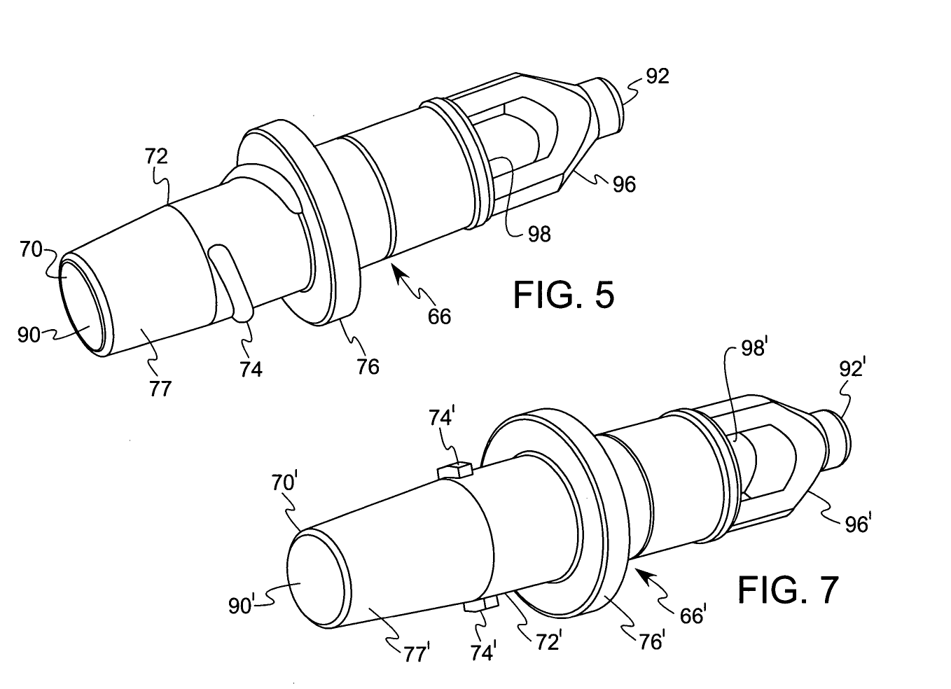

The socket 48 is adapted to engage a valve 64 in locking engagement to retain the valve 64 on the distal end 16 of the catheter body 12. In particular, the valve 64 comprises a valve body 66, a cap member 68 movably supported on the valve body 66, and an extension portion 70 at an end of the valve body 66 opposite the cap member 68 (see also FIG. 5). The extension portion 70 includes a generally cylindrical portion 72 and a locking structure illustrated as a thread member 74 extending radially outwardly from the generally cylindrical portion 72. The thread member 74 matches the configuration of the groove member 62 on the socket 48, and the extension portion 70 is inserted through the socket 48 with thread member 74 rotatably engaged in detent and recess relation with the groove member 62, providing cooperating surfaces extending transverse to the longitudinal axis 52 to retain the valve 64 on the distal end 16 of the catheter body 12. It should be noted that the outer surface of the generally cylindrical portion 72 in the area of the thread member 74 cooperates with the smooth surface of the interior wall 56 of the socket 48 to form a close fit between the surfaces of the socket 48 and the valve 64.

The valve 64 includes a radially extending flange 76 located at a mid portion of the valve body 66 between the area supporting the cap member 68 and the extension portion 70. The radially extending flange 76 engages the distal end 16 of the catheter body 12 when the valve 64 is attached to the socket 48. The extension portion 70 is formed with a length which is longer than the distance between the proximal and distal surfaces 58, 60 of the socket 48 such that the extension portion 70 extends beyond the proximal surface 58 when the valve 64 is attached with the flange 76 engaged at the distal end 16. In particular, the valve 64 is formed with a tapered portion 77 extending beyond the generally cylindrical portion 72 of the extension 70, where the tapered portion 77 is preferably formed with an 8° taper. The hub section 36 includes an expanding fluid passage 78, extending from a location adjacent the inflation port toward the socket 48, as defined by a tapered inner wall surface 80. In the attached position, the tapered portion 77 engages and stretches the material of the catheter body 12 at the tapered inner wall surface 80 of the hub section 36 where a fluid tight seal is formed between the inner wall surface 80 and the outer surface of the tapered portion 77. Thus, the hub section 36 operates to provide a positive locking connection for retaining the valve 64 to the distal end 16 of the catheter body 12 and further provides for a positive seal at the connection between the valve 64 and the catheter body 12.

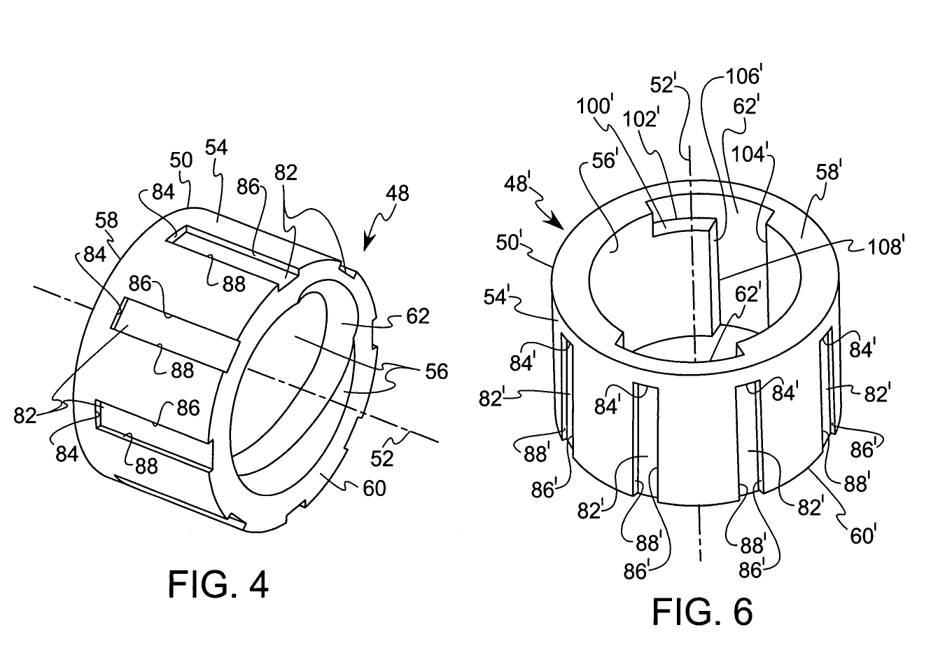

Referring to FIG. 4, the socket 48 is additionally provided with recess elements 82 formed in the exterior surface 54 to facilitate retention of the socket 48 in fixed relation to the catheter body 12. Each recess element 82 is defined by a radially oriented, laterally extending surface 84 located adjacent the end of the socket 48 defined by the proximal surface 58, and a pair of radially oriented, longitudinally extending surfaces 86, 88 which extend from the ends of the laterally extending surface 84 toward the end of the socket 48 defined by the distal surface 60. During formation of the catheter body 12, the socket 48 is molded into the catheter body 12 such that the material forming the catheter body 12 is formed into the recess elements 82. The laterally extending surfaces 84 resist movement of the socket 48 relative to the catheter body 12 in a direction parallel to the longitudinal axis 52, and the longitudinally extending surfaces 86, 88 resist movement of the socket 48 relative to the catheter body 12 in a rotational direction about the longitudinal axis 52. Accordingly, the socket 48 provides a firm attachment interface between the valve 64 and the flexible material of the catheter body 12 to avoid inadvertent detachment of the valve 64 from the catheter body 12.

Referring to FIGS. 2, 3 and 5, it should be understood that the cap member 68 of the valve 64 is linearly movable in a longitudinal direction, parallel to the longitudinal axis 52, to open and close the valve 64. The valve body 66 is formed with a hollow interior 90 and includes a central protrusion or tip 92, and the cap member 68 includes a centrally located aperture 94 engaging the tip 92 in an interference fit defining a continuous circumferential seal around the tip 92 to close the valve 64 and prevent flow of fluid and/or air through the aperture 94. The tip 92 is supported on a transition portion 96, and the transition portion 96 is provided with a pair of fluid flow openings 98, extending to either side of the valve body 66, which provide flow communication between the valve interior 90 and the interior of the cap member 68. In an open fluid flow position of the valve 64, the tip 92 is located within an in spaced relation to the cap member 68. The aperture end of the cap member 68 and the portion of the tip 92 extending through the aperture 94 are both formed with a slightly rounded configuration providing a fully exposed surface, substantially without crevices or sharp angles, which is readily wipable and which substantially avoids collection of residual fluids on the exposed surfaces of the valve 64. The disclosed valve body 66 and cap member 68 are preferably formed of a thermoplastic, such as polypropylene or an equivalent material. Additional details of the valve 64 may be found in U.S. patent application Ser. No. 10/428,431, owned by the inventors of the present application and incorporated herein by reference.

Referring to FIGS. 6 and 7, an alternative configuration of the socket-to-valve connection is illustrated, where elements of the alternative configuration corresponding to the previously described configuration are labeled with the same reference numeral primed. The socket 48′ of the alternative configuration includes a smooth interior surface 56′, including a pair of detent grooves or recesses 62′ which extend into the interior surface 56′, radially outwardly from the longitudinal axis 52′ of the socket 48′. Each detent recess 62′ includes a lateral wall 100′ located adjacent to and facing toward a proximal surface 58′ and defining a lateral groove 102′, and a pair of longitudinal walls 104′, 106′ defining a longitudinal groove 108′ extending from the location of the lateral groove 102′ to a distal end surface 60′.

The valve body 66′ includes an extension portion 70′ and a generally cylindrical portion 72′ including detent members 74′ extending from opposite sides of the valve body 66′. The detent members 74′ are adapted to pass through the longitudinal grooves 108′ as the valve body 66′ is inserted or pushed into the socket 48′, and the detent members 74′ are moved into locking engagement within the lateral grooves 102′ as the valve body 66′ is rotated or twisted to engage the detent members 74′ against the lateral walls 100′.

Referring to FIG. 8, in one aspect of the invention, the catheter 10 provides for flushing of the catheter body 12, which may be necessitated by the formation of clots in the flow path of the first passage 22, requiring occasional flushing of a fluid in the reverse direction through the first passage 22. The interior surface 56 of the socket 48 is provided with an interior diameter which is adapted to receive the tapered end 110 of a flushing syringe 112 therethrough, such that the end 110 of the syringe 112 may engage and slightly stretch the inner surface 80 of the catheter body 12. Specifically, the valve 64 may be detached from the socket 48 and the syringe 112 may be inserted to engage the end 110 of the syringe 112 with the inner wall surface 80 of the catheter body 12 to form a seal therebetween. When the flushing operation is complete, the valve 64 may be conveniently replaced in engagement with the socket 48. It should be noted that access to the end of the catheter body 12 to provide for a flushing operation is of importance in providing an unobstructed flow passage for more than a few days. The present socket attachment location provides the required access for flushing, and provides a secure removable valve attachment location for reliably retaining the valve 64 in place with a sealed connection.

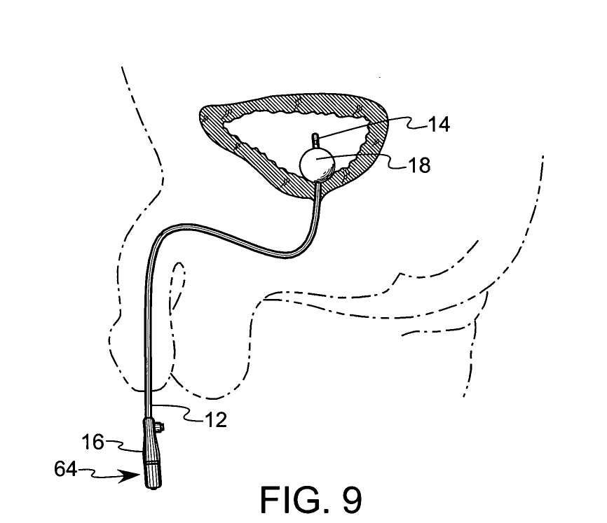

Referring to FIG. 9, which illustrates the urinary catheter 10 in relation to a partial anatomical cross-section of a male patient, it can be seen that the catheter 10 is configured to provide a compact structure having a limited extracorporal portion, providing a relatively short extension from the exit of the urethra as compared to prior art catheter systems incorporating a collection bag. For example, the extracorporal portion of the catheter body 12 may comprise a length of one to two inches or more, which may be easily contained in the undergarments of the patient.

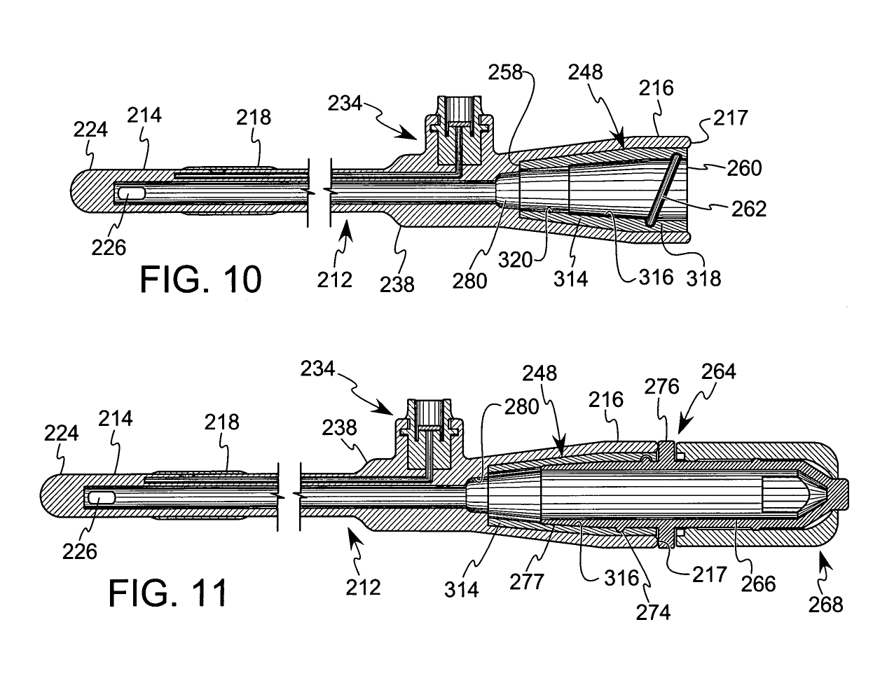

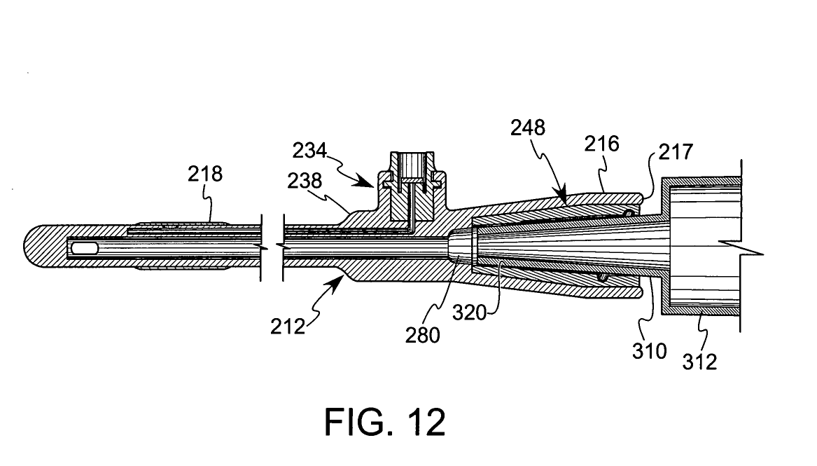

Referring to FIGS. 10-12, an alternative embodiment illustrating the invention is shown where elements corresponding to elements of the first described embodiment are labeled with the same reference numeral increased by 200. As may best seen in FIG. 10, the present embodiment provides a socket 248 having an elongated proximal section 314 where the socket 248 is molded within the catheter body 212. It should be noted that the distal end 216 of the catheter body 212 is formed with a sealing bead defined by a radiused edge 217 extending in the longitudinal direction beyond the distal end surface 260 of the socket 248.

The interior of the socket 248 along the elongated proximal section 314 is formed with a first tapered surface 316 located adjacent a distal section 318 containing a radially extending locking structure, such as a groove defining a recessed thread 262. The first tapered surface 316 angles inwardly, in a direction from the distal section 318 toward the proximal end 258 of the socket 248, at an angle of approximately 8° for receiving and engaging the tapered end 277 of the valve 264, where a sealing engagement is formed between the tapered end 277 of the valve 264 and the first tapered surface 316, as may be seen in FIG. 11. When the valve 264 is located in position within the socket 248, the radially extending flange 276 engages the radiused edge 217 in sealing engagement to prevent leakage of fluid between the valve 264 and the distal end 216 of the catheter body 212.

The interior of the socket 248 is further formed with a second tapered surface 320 extending from the first tapered surface 316 to the proximal end 258. The second tapered surface 320 is tapered inwardly at an angle of approximately 8° and is separated from the first tapered surface 316 by a step such that the diameter of the second tapered surface 320 is smaller than the diameter of the first tapered surface 316. The second tapered surface 320 is adapted to receive and engage the tapered end 310 of a conventional flushing syringe 312, with the outer surface of the syringe end 310 located in generally circumferentially spaced relation to the first tapered surface 316. A sealing engagement is defined between the second tapered surface 320 and the syringe end 310.

It should be understood that the locking structure for maintaining the valve 264 in engagement with the socket 248 may be formed with other configurations than the illustrated recessed thread 262 for engaging a cooperating thread member 274 of the valve 264. For example, the locking structure may be formed in accordance with the configuration of FIGS. 6 and 7, providing a push and twist engagement between the valve 264 and the socket 248.

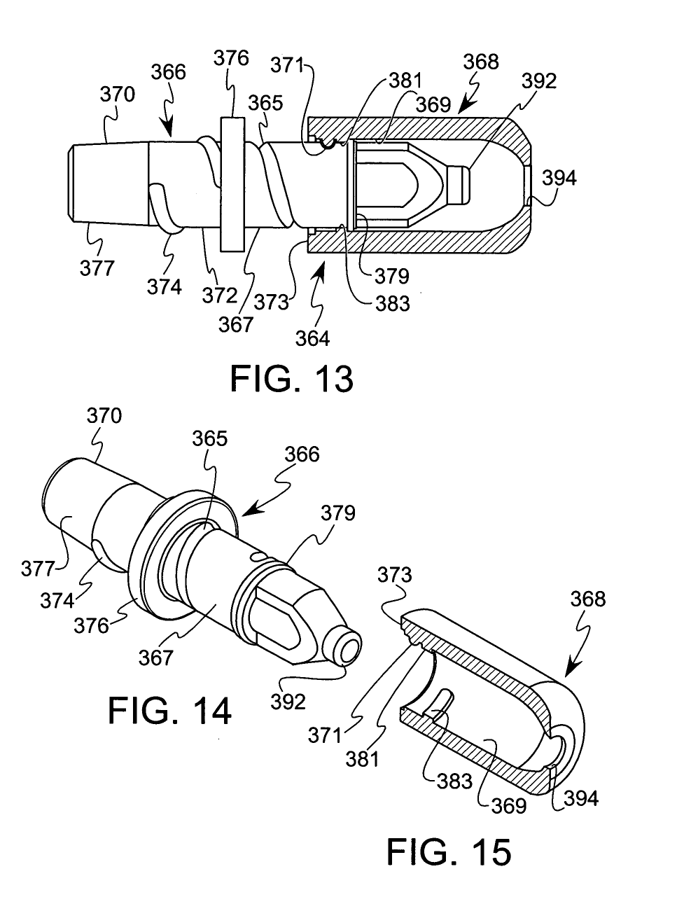

Referring to FIGS. 13-15, an alternative embodiment of a valve for use in the present catheter system is illustrated, and in which elements corresponding to the elements described with reference to the embodiment of FIG. 3 are identified with the same reference numeral increased by 300.

The valve 364 illustrated in FIG. 13 is shown in an open flow position and includes a valve body 366 and a cap member 368. The valve body 366 includes a thread groove 365 defined as a spiral groove extending around the valve body 366 on a surface 367 of the valve body 366 provided for engaging and supporting a portion of the cap member 368. An inner surface 369 of the cap member 368 is formed with a thread post 371 which engages in the thread groove 365, such that rotation of the cap member 368 relative to the valve body 366 causes the cap member 368 to move longitudinally relative to the valve body 366. Rotation of the cap member 368 in a first direction causes the tip 392 to sealingly engage the aperture 394, and the end 373 of the cap member 368 engages the flange 376 to limit movement of the cap member 368 and defines the closed position. Rotation of the cap member 368 in an opposite second direction causes the aperture 394 to move to an open position out of engagement with the tip 392.

A pair of position stops 381, 383 are located on opposing sides of the inner surface 369 of the cap member 368 and engage against a radially extending skirt 379 on the valve body 368. The engagement of position stops 381, 383 against skirt 379 limits movement of the cap member 368 and defines the open position for the valve 364. In addition, the skirt 379 cooperates with the inner surface 369 of the cap member 368 in a close fit to provide a sliding seal therebetween to prevent migration of fluid from the interior of the cap member 368 toward the open end 373.

As described above with regard to the valve 64, the present valve 364 provides an easily wipable surface when the valve 364 is closed. Specifically, the valve 364 provides an inner closure member tip 392 which moves through the aperture 394 of the surrounding cooperating cap member 368 to position the tip 392 in an exposed position for cleaning or wiping with the outer surface of the cap member 368 to reduce the possibility of growth of bacteria from exposed residual fluids.

While the forms of apparatus herein described constitute preferred embodiments of this invention, it is to be understood that the invention is not limited to these precise forms of apparatus, and that changes may be made therein without departing from the scope of the invention which is defined in the appended claims.

What is claimed is:

1. A urinary catheter comprising:
an elongated tube formed of a flexible material;
a proximal end of said tube formed by the flexible material and including an opening placing an exterior of said elongated tube in fluid communication with an interior fluid passage of said elongated tube, said proximal end defining an inlet end of said elongated tube;
a distal end of said tube formed by said flexible material;
a socket located within said distal end of said flexible material and defining a fluid outlet for said elongated tube; and
a valve having a fluid passage therethrough and connected to said socket, said valve including a closure member displaceable to control fluid flow from said elongated tube.

2. The urinary catheter of claim 1 wherein said socket includes an interior surface and said valve includes a valve extension defining an exterior surface cooperating with said interior surface of said socket.

3. The urinary catheter of claim 2 wherein said interior surface of said socket includes a portion defining a first radially extending locking structure, and said exterior surface of said valve extension defines a second radially extending locking structure cooperating with said first locking structure.

4. The urinary catheter of claim 3 wherein said first and second locking structures comprise cooperating thread members defined on said socket and said exterior surface of said valve extension.

5. The urinary catheter of claim 3 wherein said first and second locking structures comprise cooperating detents and grooves.

6. The urinary catheter of claim 2 wherein said socket includes a proximal end and a distal end, and said valve extension extends past said proximal end into engagement with said flexible material to define a fluid seal.

7. The urinary catheter of claim 1 wherein said distal end of said tube extends beyond said socket, and said valve includes a radially extending flange portion cooperating with said distal end of said tube to define a fluid seal.

8. The urinary catheter of claim 1 wherein said elongated tube comprises a secondary passage to define a double lumen catheter, and a valve port located adjacent said distal end defined by said flexible material and in fluid communication with said secondary passage, and said proximal end of said tube including an expandable balloon element formed in said flexible material and in fluid communication with said secondary passage.

9. The urinary catheter of claim 1 wherein a sealing surface of a first member of said valve for cooperating with a second member of said valve for controlling fluid flow comprises an outer surface of said valve to facilitate wiping of the area of the sealing surface.

10. The urinary catheter of claim 9 wherein said second member is supported internally of and in spaced relation to said first member during fluid flow, and at least a portion of said second member extends outwardly from said first member to facilitate wiping said second member when said valve is closed.

11. The urinary catheter of claim 1 wherein said valve comprises a generally cylindrical valve body and a generally cylindrical, movable closure member attached to said valve body, said movable closure member including a central aperture defining a valve outlet and said valve body including a central protrusion for engaging within said central aperture to define a closure for said valve, said closure member longitudinally displaceable between open and closed positions on said valve body to control fluid flow from said elongated tube.

12. The urinary catheter of claim 11 wherein said movable closure member is movable in linear movement relative to said valve body.

13. The urinary catheter of claim 11 wherein at least one of said movable closure member and said valve body include a thread element to guide said movable closure member in longitudinal movement in response to rotation of said closure member.

14. The urinary catheter of claim 2 wherein said socket includes a first tapered surface engaging said extension of said valve, and a second tapered surface adjacent said first tapered surface defining a diameter smaller than a diameter defined by said first tapered surface.

15. A urinary catheter comprising:
an elongated tube formed of a flexible material;
a proximal end of said tube formed by the flexible material and including an opening placing an exterior of said elongated tube in fluid communication with an interior fluid passage of said elongated tube, said proximal end defining an inlet end of said elongated tube;
a distal end of said tube formed by said flexible material;
a secondary passage extending parallel to said fluid passage;
an actuated element in fluid communication with said secondary passage;
a socket located within said distal end of said flexible material and defining a relatively rigid fluid outlet for said elongated tube.

16. The urinary catheter of claim 15 wherein said socket comprises an interior surface and a first locking structure extending radially from a portion of said interior surface.

17. The urinary catheter of claim 16 wherein said first locking structure extends radially outwardly from said interior surface, and said interior surface defines a substantially smooth surface.

18. The urinary catheter of claim 16 including a valve having a fluid passage therethrough, and having an exterior surface and a second locking structure extending radially from said exterior surface and cooperating with said first locking structure.

19. The urinary catheter of claim 15 including a valve having a fluid passage therethrough and including a tapered extension engaged with a first tapered surface of said socket.

20. The urinary catheter of claim 19 including a second tapered surface defined in said socket adjacent said first tapered surface, said second tapered surface separated from said first tapered surface by a step.

21. The urinary catheter of claim 15 wherein said socket comprises an exterior surface in engagement with said flexible material, said exterior surface including grooves interengaged with said flexible material.

22. The urinary catheter of claim 15 including a valve port in fluid communication with said secondary passage and located adjacent said distal end of said elongated tube, and said actuated element comprising a balloon located at said proximal end in fluid communication with said valve port.

23. The urinary catheter of claim 15 including a valve detachably attached to said socket and having a fluid passage therethrough, said valve including a valve body and a movable closure member attached to said valve body and displaceable between open and closed positions to control fluid flow from said elongated tube.

24. The urinary catheter of claim 15 including a valve detachably attached to said socket for controlling fluid flow through said catheter, and including a sealing bead adjacent an outer surface of said socket for engaging a surface of said valve in sealing engagement.
